(19) United States
(12) Patent Application Publication
(10) Pub. No.: US 2020/0338341 A1
(43) Pub. Date: Oct. 29, 2020

(54) HEART CATHETER

(71) Applicant: VM HOLDING GMBH, Heidelberg (DE)

(72) Inventor: Viktor Moritz Markstein, Heidelberg (DE)

(21) Appl. No.: 16/959,797

(22) PCT Filed: Jan. 7, 2019
(86) PCT No.: PCT/EP2019/050245

§ 371 Date: Jul. 2, 2020

(30) Foreign Application Priority Data
Jan. 5, 2018 (DE) 10 2018 100 199.1

(51) Int. Cl. A61N 1/05; A61B 90/00

(52) U.S. Cl. CPC A61N 1/0565; A61B 2018/00577; A61B 90/39

(57) ABSTRACT

A cardiac catheter having electrodes, including a number of elastic individual carriers, which are each connected to a distal end region of the catheter, wherein the carriers each have a longitudinal axis, a number of electrodes per carrier, wherein the electrodes are arranged in lines on an outside of the respective carrier, the lines extend perpendicular to the longitudinal axis, and a mechanism configured so that the carriers, after an insertion of the distal end region of the cardiac catheter into a subspace of a heart, are capable of being reversibly brought from a first state into a second state, wherein the mechanism spreads out the carriers in the first state such that the electrodes are each applied to an inner surface of the subspace, and the mechanism closes the carriers in the second state such that the catheter having the carriers is removable from the subspace.

### CROSS REFERENCE TO RELATED APPLICATIONS

This application is the U.S. National Phase of, and Applicant claims priority from, International Patent Application No. PCT/EP2019/050245, filed on 7 Jan. 2019, and German Patent Application No. DE 10 2018 100 199.1, filed on 5 Jan. 2018, both of which are incorporated herein by reference in their entirety.

### FIELD

The invention relates to a cardiac catheter having electrodes arranged at the distal end of the cardiac catheter. The proposed cardiac catheter is particularly suitable for examining and treating atrial fibrillations and ventricular arhythmias.

The object of the invention is to provide a cardiac catheter using which, in particular, electrophysical/electrophysiological processes in the tissue of the heart muscle, which occur, for example, during atrial fibrillation, can be recorded more precisely.

The invention results from the features of the independent claims. Advantageous refinements and designs are the subject matter of the dependent claims. Other features, possible applications, and advantages of the invention will be apparent from the following description, and from the explanation of example embodiments of the invention, which are illustrated in the figures.

A first aspect of the invention relates to a cardiac catheter having electrodes, including a number N of elastic individual carriers Tₙ with n=1, 2, . . . , N and Nϵ{2, 3, . . . , 10}, which are each connected to a distal end region of the cardiac catheter, wherein the carriers Tₙ each have a longitudinal axis LAₙ (along their respective longest extension), a number Mₙ of electrodes E_{n,m} per carrier Tₙ, with m=1, 2, . . . , Mₙ and Mₙ≥5, wherein the electrodes E_{n,m} are arranged in lines R_{k,m,n} on an outside of the respective carrier Tₙ, with k=1, 2, . . . K and K≥3, the lines R_{k,m,n} extend perpendicular to the longitudinal axis LAₙ, and furthermore include a mechanism configured so that the carriers Tₙ, after an insertion of the distal end region of the cardiac catheter into a subspace of a heart, are capable of being brought reversibly from a first state into a second state, wherein the mechanism spreads out the carriers Tₙ in the first state such that the electrodes E_{n,m} are each applied to an inner surface of the subspace, and the mechanism closes the carriers Tₙ in the second state such that the catheter having the carriers Tₙ is removable from the subspace.

The term “carrier” Tₙ is understood broadly in the present case. In the present case, a carrier Tₙ serves as a physical holder for the electrodes E_{n,m}. It is essential that the carriers Tₙ are designed such that the electrodes E_{n,m} are arranged in a plurality of rows R_{k,m,n} oriented orthogonally to the respective longitudinal axis LAₙ of the individual carriers Tₙ It is also essential that the cardiac catheter has at least two or more separate carriers Tₙ, wherein each of the carriers Tₙ has its own connection to the distal end region of the cardiac catheter. The carriers Tₙ are designed to be elastic, so that, on the one hand, they can be brought reversibly into the first and the second state and, on the other hand, in the first state they also carry out movements of the inner surface of the subspace (with a beating heart) due to their elasticity, wherein the electrodes E_{n,m} optimally each remain at a respective position on the inner surface. In this way, electrical potentials can be measured by the electrodes E_{n,m} from the respective associated point of the inner surface of the subspace.

In one advantageous refinement of the proposed cardiac catheter, the carriers Tₙ are detachably connected fixedly to the distal end region of the cardiac catheter. This enables the individual carriers Tₙ to be removed, for example, to sterilize the carriers after an intervention and/or to install the carriers Tₙ with a different number of electrodes E_{n,m} and/or a different shaping on the cardiac catheter.

One advantageous refinement of the proposed cardiac catheter is distinguished in that at least one of the carriers Tₙ is connected to the cardiac catheter at a different longitudinal position along a longitudinal axis of the cardiac catheter than the remaining carriers Tₙ This enables the carriers Tₙ to be applied to the inner surface of the cavity in different regions along the cardiac catheter.

The carriers Tₙ on the catheter are advantageously each at an identical distance from the distal end of the catheter on the catheter. The carriers Tₙ are advantageously arranged on the catheter evenly distributed along a circumferential line of the catheter.

Advantageously, an end piece of the catheter projecting distally from the connections of the carriers Tₙ to the catheter includes an ablation device and/or an injection device and/or a device for taking samples. This embodiment makes it possible to determine appropriate electrical signals via the carriers Tₙ and to identify points on the inner surface of the cavity to be treated on the basis of the signals. Furthermore, it is possible to carry out targeted treatments in the cavity of the heart on the inner surface regions by the devices mentioned, which are arranged at the distal end of the catheter.

One particularly advantageous refinement of the proposed cardiac catheter is distinguished in that the carriers Tₙ each include at least three markers of a position acquisition system. This enables the 3D positions of the at least three markers to be acquired. The positions of the electrodes E_{n,m} can be estimated on the basis of these ascertained marker positions. The (3D) position estimation of the electrodes E_{n,m} improves the more markers are present. A carrier Tₙ advantageously includes a number of 3 to 20 markers, in particular 5 to 15 markers.

Depending on the position acquisition system used (e.g., 3D ultrasound, computer tomography (CT), fluoroscopy, magnetic resonance tomography (MRT), DEMRI (delayed enhanced MRI (Magnetic Resonance Imaging), etc.), the markers are embodied in such a way that they are precisely detectable by the respective position acquisition system and their respective position is precisely ascertainable. The markers are, for example, gas bubbles or elements made of metal/metal alloys or elements having different shaped segments or elements having different segments made of different materials. It is advantageous that the size of the markers is optimized so that on the one hand they are as small as possible (which enables a higher positional accuracy), but on the other hand they are sufficiently large that unique detectability of the markers by the position acquisition system is ensured.

In one preferred embodiment of the cardiac catheter, the carriers Tₙ each have a flat shape and consist of an elastic polymer-based material, in particular of a silicone-based material. The carriers Tₙ each advantageously include a flat elastic membrane MEM, which is connected to a reticular support structure. The elastic membrane MEM is advantageously arranged flat between the reticular support structure. The support structure is preferably used for a variably adjustable shaping of the membrane MEM. The electrodes E_{n,m} are advantageously arranged on the outside of the membrane MEM. In one development, the support structure is finger-shaped, that is to say it essentially includes linear support structure elements starting from one point and extending outwards. In a further advantageous embodiment of the cardiac catheter, the support structure includes a plurality of longitudinal beams, which are preferably connected to one another centrally at the ends, so that the longitudinal beams extend radially from the central connection. The longitudinal beams are advantageously connected to the catheter at their respective opposite ends. The central connection can advantageously be mechanically displaced in the longitudinal direction in a predetermined region of the catheter, so that when the distance between the central connection and the connections of the opposite ends of the longitudinal beams to the catheter is reduced, the longitudinal beams bulge oriented radially away from the catheter. The support structure advantageously consists of a shape memory alloy. In the first state, the support structure advantageously assumes a shape which presses an elastic membrane connected to the support structure with the electrodes E_{n,m} against the inner surface of the subspace with a predetermined force.

Advantageously, the carriers Tₙ have a leaf-like, in particular a beech-leaf-like or olive-leaf-like shape or delimitation, that is to say that the carriers Tₙ each have a large lateral extension with respect to the respective longitudinal axis LAₙ starting from their roots (i.e., the region of the carrier Tₙ which is connected to the catheter), which decreases with increasing distance from the root along the longitudinal axis LAₙ.

The carriers Tₙ each advantageously have a supporting structure which consists of a shape memory alloy, in particular nitinol. A first shape is advantageously applied to the support structure. This first shape is advantageous in such a way that the respective carriers Tₙ are pressed flat against the inner surface of the subspace with a predetermined force in the first state.

The elastic membrane MEM advantageously has openings through which blood can flow from one side of the membrane MEM to the opposite side. This can ensure that the side of the membrane with electrodes E_{n,m} arranged thereon can be applied to the inner surface of the cavity as optimally as possible and that no local blood bubbles form which prevent the electrodes E_{n,m} from being applied.

For contacting the electrodes E_{n,m} there are advantageously first electrical lines within the carrier Tₙ for the individual contacting of the respective electrodes E_{n,m}, which are made of an electrically conductive, elastic polymer-based material. This ensures that the first electrical lines elastically follow expansions and compressions of the carriers Tₙ. The elastic design of the first electrical lines prevents line breaks, as they occur with metal lines.

One advantageous refinement of the cardiac catheter is characterized in that the carriers Tₙ each have an interface for the electrically conductive connection of the first lines to second electrical lines made of a metal or a metal alloy. The second lines advantageously extend from the interface to a proximal end of the catheter.

Overall, the cardiac catheter has a number

$${\sum }_{n = 1}^{N} {M}_{n}$$

of electrodes E_{n,m}. Depending on the metrological implementation, the electrical signals acquired by the electrodes are advantageously first conducted via the first elastic lines to the above-mentioned interface. In one variant, at least

$${\sum }_{n = 1}^{N} {M}_{n}$$

individual second (signal) lines made of metal (in particular made of gold, platinum) are led from the interface to the proximal end of the catheter. The second lines are advantageously electrically insulated from one another and individually electrically shielded.

In another variant, the

$${\sum }_{n = 1}^{N} {M}_{n}$$

second lines coming from the interface are led to an electronic unit which is arranged in the distal end region of the catheter, and which generates a digital data stream from the

$${\sum }_{n = 1}^{N} {M}_{n}$$

analog measurement signals of the electrodes E_{n,m} by digitizing and multiplexing, which is conducted via a data line running in the catheter to the proximal end of the catheter.

At the proximal end of the catheter, the signals or data provided there are fed to a data processing device.

In one advantageous refinement of the proposed cardiac catheter, the carriers Tₙ each include a flat elastic membrane MEM, which includes the following three elastic layers: —an upper layer on which the electrodes E_{n,m} are arranged,—a lower layer, and—a middle layer in which electrical lines extend.

Spacers are advantageously arranged in the middle layer in order to space apart the upper layer and lower layer at a distance A. The middle layer is also advantageously designed such that a volume through which a fluid can flow is present between the upper layer and the lower layer and the spacers. The upper layer, the lower layer, and also the spacers are advantageously manufactured from an elastic polymer material. A circumferential edge of the elastic membrane MEM is advantageously closed in a fluid-tight manner, wherein at least one fluid channel for the inflow or outflow of fluid into or out of the middle layer is provided for the fluid supply or fluid discharge, respectively, in the middle layer.

This at least one fluid channel is advantageously provided with a controllable device for filling and emptying with/from fluid the volume of the middle layer through which fluid can flow.

The number Mₙ of the electrodes E_{n,m} present on a carrier Tₙ is advantageously Mₙ≥10, Mₙ≥20, Mₙ≥30, Mₙ≥40, Mₙ≥50, Mₙ≥60, Mₙ≥70, Mₙ≥80, Mₙ90, Mₙ≥100 or M≤12 or Mₙ=42.

The following advantageously applies for the number N of carriers Tₙ arranged at the distal end region of the catheter: N=3 or N=5. The following advantageously applies for the number K of the lines R_{k,m,n} of electrodes E_{n,m} arranged on a carrier Tₙ: Kε{3, 4, . . . , 20}.

As indicated, the mechanism is used to bring the carriers Tₙ reversibly from a first state into a second state after an insertion of the distal end region of the cardiac catheter into a subspace of a heart, i.e., on the one hand, to unfold the individual carriers Tₙ in the subspace of the heart such that the electrodes E_{n,m} are applied as unchangeably as possible at the respective positions on the inner surface of the cavity even with a beating heart (first state), and, on the other hand, to reduce the volume requirement of the individual carriers Tₙ in such a way that an uncomplicated discharge or introduction into the subspace of the heart through the catheter is possible.

For this purpose, the mechanism is advantageously coupled to a pneumatic and/or hydraulic and/or electrical and/or magnetic actuator and/or a temperature-controllable fluid source in such a way that the carriers Tₙ can be brought reversibly into the first and second state by a corresponding activation or deactivation of the actuator.

The mechanism is particularly advantageously embodied and configured such that the carriers Tₙ having the electrodes E_{n,m} arranged thereon are pressed against the inner surface of the subspace in the first state with a predetermined contact force. Furthermore, the mechanism is advantageously embodied and configured such that this contact force remains essentially constant regardless of the time even with an inner surface that contracts or widens when the heart beats.

The mechanism advantageously includes at least one elastic structural element or at least one elastic membrane element which can be filled with a fluid.

The filling with fluid advantageously takes place at a controllable variable pressure. The fluid pressure is advantageously controlled in dependence on a movement (contraction/expansion) of the inner surface of the cavity. The movement of the cavity can be ascertained, for example, by ultrasound measurement. Furthermore, the movement of the cavity can be predicted from existing measurement data. The mechanism is in particular designed in such a way that the carriers Tₙ are each pressed flat against the inner surface of the subspace with an essentially constant contact force. The mechanism thus creates a kind of counterforce to a contraction or relaxation force of the heart muscle forming the inner surface of the cavity. This counterforce can be used in particular to acquire, for example, the local force input transmitted from the heart muscle to the respective carrier Tₙ. For this purpose, the carriers Tₙ are advantageously equipped with a sensor system which makes it possible to acquire a local force input. Corresponding sensors are known in the prior art, for example from Tacterion GmbH.

A particularly advantageous embodiment variant of the cardiac catheter is distinguished in that: N=3 and the angles between the three longitudinal axes LAₙ of the carriers Tₙ are each 120°, at least insofar as they are projected onto a plane, so that the longitudinal axes LAₙ intersect at an intersection.

One advantageous refinement of the cardiac catheter is distinguished in that the electrodes E_{n,m} have at least two different sizes of contact surfaces.

The electrodes E_{n,m} are applied with their contact surfaces to the inner surface of the subspace of the heart in the first state.

One advantageous refinement of the cardiac catheter is distinguished in that the electrodes E_{n,m} have at least two different sizes G1 and G2 of contact surfaces, wherein: G2≥2*G1.

One advantageous refinement of the cardiac catheter is distinguished in that the electrodes E_{n,m} have at least two different shapes of contact surfaces. The electrodes E_{n,m} advantageously have two different shapes F1 and F2 of contact surfaces, wherein the following is true: the shape F1 is circular and the shape F2 has a longitudinal extension which is greater than a perpendicular extension of the shape F2.

A first set of the electrodes E_{n,m} advantageously consists of a first material and a second set of the electrodes consists of a second material different therefrom. For example, the first set consists of gold electrodes and the second set consists of platinum electrodes.

One advantageous refinement of the proposed cardiac catheter is characterized in that the electrodes E_{n,m} of each individual line R_{k,m,n} have identical sizes of contact surfaces, wherein the sizes of contact surfaces of the electrodes E_{n,m} of successive lines R_{k,m,n} alternate between two sizes of the contact surfaces, i.e., the sizes G1 and G2 of the electrodes of successive lines alternate as follows: R_{k=1,m,n}G1, R_{k=2,m,n}=G2, R_{k=3,m,n}=G1, . . . .

A distance between two adjacent electrodes E_{n,m} of a line R_{k,m,n} is advantageously constant for all electrodes E_{n,m} of this line R_{k,m,n}. The electrodes E_{n,m} advantageously consist of a metal or a metal alloy or of an electrically conductive polymer-based material or of carbon.

The electrodes E_{n,m} are used, on the one hand, to decrease electrical potentials, on the other hand, all or some selected electrodes E_{n,m} can also be used for the local application of current or voltage surges. The electrodes E_{n,m} are controlled accordingly for this purpose. In the region of its distal end, the catheter advantageously includes one or more electrodes, which are designed to deliver local electrical stimuli into the inner surface of the subspace.

Each carrier Tₙ advantageously includes an electrical connecting element which is connected, on the one hand, via M electrical lines to the individual electrodes E_{n,m} of the carrier Tₙ, and on the other hand, is connectable to an evaluation unit via one or more electrical lines extending in the catheter. The connecting element advantageously includes an electrical amplifier and/or multiplexer and/or electrical filter.

At least one of the carriers Tₙ advantageously includes one or more additional sensors for acquiring one or more additional physical or chemical or biological parameters. The additional physical or chemical or biological parameter is advantageously a mechanical force and/or a mechanical torque acting externally on the carrier Tₙ, in particular from the inner surface of the subspace, a temperature, a concentration of a specified substance, a light signal, in particular a fluorescence signal, or a parameter dependent thereon.

Multiple additional sensors of this type are advantageously provided on at least one of the carriers Tₙ. These sensors are advantageously arranged in such a way that a planar distribution of the respective parameter or parameters can be acquired. The measurement signals acquired by these sensors are advantageously conducted in the respective carrier Tₙ by elastic electrical lines to an interface which is advantageously arranged at the root of the respective carrier Tₙ. From there, at least one electrical line (advantageously a metallic electrical line) is led to the proximal end of the catheter and provides sensor signals from the sensors for an evaluation unit there.

One particularly advantageous refinement relates to a cardiac catheter with electrodes, including a number N of elastic individual carriers Tₙ with n=1, 2, . . . , N and Nϵ{2, 3, . . . , 10}, wherein the carriers Tₙ each include an elastic longitudinal beam LTₙ, the proximal end of which connects the carrier Tₙ to a distal end region of the cardiac catheter and the distal end of which is a free end, a longitudinal axis LAₙ, extending along the respective longitudinal beam LTₙ, a number Kₙ of elastic cross beams QT_{n,k} and a number Mₙ of electrodes E_{n,m} with m=1, 2, . . . Mₙ and Mₙ≥5, wherein the electrodes E_{n,m} are arranged on a respective outside of the respective cross beam QT_{n,k} with k=1, 2, . . . , K and Kₙ≥3, and a mechanism configured so that the carriers Tₙ, after an insertion of the distal end region of the cardiac catheter into a subspace of a heart, can be reversibly brought from a first state into a second state, wherein the mechanism spreads out or unfolds the carriers Tₙ in the first state such that the electrodes E_{n,m} are each applied to an inner surface of the subspace, the cross beams QT_{n,k} extend perpendicular to the respective longitudinal beam LTₙ and/or perpendicular to the longitudinal axis LAₙv and the mechanism closes the carriers Tₙ in the second state such that the catheter having the carriers Tₙ is removable from the subspace.

The cross beams QT_{n,k} are advantageously arranged on the associated longitudinal member LTₙ such that they extend (in the first state) equal distances perpendicularly to the longitudinal beam LTₙ on two sides of the longitudinal beam LTₙ The cross beams QT_{n,k} on the associated longitudinal beam LTₙ are advantageously arranged equidistant from one another. The cross beams QT_{n,k} on the associated longitudinal beam LTₙ can advantageously be arranged at an adjustable distance from one another.

The longitudinal beam LTₙ and/or the cross beams QT_{n,k} advantageously consist of a shape memory alloy and are conditioned accordingly in such a way that they assume the specified geometry in relation to one another in the first state.

In an alternative refinement, the cross beams QT_{n,k} and/or the longitudinal beam LTₙ include fluid chambers, which can be individually filled with fluid or emptied of fluid. The cross beams QT_{n,k} and/or the longitudinal beam LTₙ is embodied such that, when the fluid chamber is filled with a fluid, they assume a predetermined pose in which, as stated above, the cross beams QT_{n,k} extend perpendicularly to the longitudinal beam LTₙ and/or perpendicularly to the longitudinal axis LAₙ.

The carriers Tₙ are advantageously arranged on the catheter evenly distributed along a circumferential line of the catheter. Advantageously, an end piece of the catheter projecting distally from the connections of the carriers Tₙ to the catheter includes an ablation device and/or an injection device and/or a device for taking samples. This device/these devices is/are advantageously guided centrally in the catheter and is/are embodied to be longitudinally displaceable in the axial direction of the catheter. This embodiment makes it possible to determine appropriate electrical signals via the carriers Tₙ and to identify points on the inner surface of the cavity to be treated on the basis of the signals. Furthermore, it is possible to carry out targeted treatments in the cavity of the heart on the inner surface regions by the devices mentioned, which are arranged at the distal end of the catheter.

One particularly advantageous refinement of the proposed cardiac catheter is distinguished in that the carriers Tₙ each include at least three markers of a position acquisition system. This enables the 3D positions of the at least three markers to be acquired. The positions of the electrodes E_{n,m} can be estimated on the basis of these ascertained marker positions. The (3D) position estimation of the electrodes E_{n,m} improves the more markers are present. A carrier Tₙ advantageously includes a number of 3 to 20 markers, in particular 5 to 15 markers.

Depending on the position acquisition system used (e.g., 3D ultrasound, computer tomography (CT), fluoroscopy, magnetic resonance tomography (MRT), DEMRI (delayed enhanced MRI (magnetic resonance imaging), etc.), the markers are embodied in such a way that they are precisely recognizable by the respective position acquisition system and their respective position is precisely ascertainable. The markers are, for example, gas bubbles or elements made of metal/metal alloys or elements having different shaped segments or elements having different segments made of different materials. It is advantageous that the size of the markers is optimized so that on the one hand they are as small as possible (which enables a higher positional accuracy) but on the other hand they are sufficiently large that unique recognizability of the markers by the position acquisition system is ensured.

One advantageous refinement of the cardiac catheter is distinguished in that the carriers Tₙ each have an interface for the electrically conductive connection of the first lines to second electrical lines made of a metal or a metal alloy. The second lines advantageously extend from the interface to a proximal end of the catheter.

Overall, the cardiac catheter has a number

$${\sum }_{n = 1}^{N} {M}_{n}$$

of electrodes E_{n,m}. Depending on the metrological implementation, the electrical signals acquired by the electrodes are advantageously first conducted via the first elastic lines to the above-mentioned interface. In one variant, at leas

$${\sum }_{n = 1}^{N} {M}_{n}$$

individual second (signal) lines made of metal (in particular made of gold, platinum) are led from the interface to the proximal end of the catheter. The second lines are advantageously electrically insulated from one another and individually electrically shielded.

In another variant,

$${\sum }_{n = 1}^{N} {M}_{n}$$

second lines coming from the interface are led to an electronic unit which is arranged in the distal end region of the catheter, and which generates a digital data stream from the

$${\sum }_{n = 1}^{N} {M}_{n}$$

analog measurement signals of the electrodes E_{n,m} by digitizing and multiplexing, which is conducted via a data line running in the catheter to the proximal end of the catheter.

At the proximal end of the catheter, the signals or data provided there are fed to a data processing device.

The number Mₙ of the electrodes E_{n,m} present on a carrier Tₙ is advantageously Mₙ≥10, Mₙ≥20, Mₙ≥30, Mₙ≥40, Mₙ≥50, Mₙ≥60, Mₙ≥70, Mₙ≥80, Mₙ≥90, Mₙ≥100 or M≤12 or Mₙ=42.

The following advantageously applies for the number N of carriers Tₙ arranged at the distal end region of the catheter: N=3 or N=5. The following advantageously applies for the number Kₙ of the cross beams QT_{n,k} arranged on a carrier Tₙ: Kε{3, 4, . . . , 20}.

As indicated, the mechanism is used to bring the carriers Tₙ reversibly from a first state into a second state after an insertion of the distal end region of the cardiac catheter into a subspace of a heart, i.e., on the one hand, to unfold the individual carriers Tₙ in the subspace of the heart such that the electrodes E_{n,m} are applied as unchangeably as possible at the respective positions on the inner surface of the cavity even with a beating heart (first state), and, on the other hand, to reduce the volume requirement of the individual carriers Tₙ in such a way that an uncomplicated discharge or introduction into the subspace of the heart through the catheter is possible.

For this purpose, the mechanism is advantageously coupled to a pneumatic and/or hydraulic and/or electrical and/or magnetic actuator and/or a temperature-controllable fluid source in such a way that the carriers Tₙ can be brought reversibly into the first and second state by a corresponding activation or deactivation of the actuator. Alternatively or additionally, the mechanism is embodied so it is mechanically operable by an operator.

The mechanism is particularly advantageously designed and configured such that the carriers Tₙ having the electrodes E_{n,m} arranged thereon are pressed against the inner surface of the subspace in the first state with a predetermined contact force. Furthermore, the mechanism is advantageously designed and configured such that this contact force remains essentially constant regardless of the time even with an inner surface that contracts or expands when the heart beats.

In one advantageous refinement, the carriers Tₙ each include a flat elastic membrane MEM, which connects the cross beams QT_{n,k} and the longitudinal beams LTₙ of the respective carrier Tₙ. The flat elastic membrane MEM advantageously has the following three elastic layers: an upper layer, on which the electrodes E_{n,m} are arranged, a lower layer, and a middle layer, in which electrical elastic first lines for contacting the electrodes E_{n,m} extend, wherein spacers are arranged in the middle layer for spacing apart the top layer and bottom layer at a distance A, and the middle layer has a volume through which a fluid can flow.

The longitudinal beams LTₙ are particularly advantageously connected to the distal end region of the cardiac catheter in such a way that they are rotatable. This enables, in particular, an individual rotation of each individual one of the carriers Tₙ and thus an adjustable configuration or relative arrangement of the rows of electrodes or cross beams QT_{n,k} arranged on the individual carrier Tₙ in relation to one another. The rotation can be done, for example, by mechanical operation by an operator and/or by a controlled actuator.

The carriers Tₙ advantageously have a number of Mₙ first electrical lines for the individual contacting of the respective electrodes E_{n,m}, which are made of an electrically conductive, elastic polymer-based material.

Particularly advantageously, the mechanism has an elastic inflatable body (e.g., a balloon body), which can be filled and emptied, and which can be filled with a fluid in the first state and thereby presses it outward from an inside of the respective cross beam QT_{n,k}. The inflatable body is advantageously supplied with fluid through the catheter.

The filling of the bladder body with fluid is advantageously carried out using a controllable variable pressure. The fluid pressure is advantageously controlled in dependence on a movement (contraction/expansion) of the inner surface of the cavity. The movement of the cavity can be ascertained, for example, by ultrasound measurement. Furthermore, the movement of the cavity can be predicted from existing measurement data. The mechanism is in particular designed in such a way that the carriers Tₙ are each pressed flat against the inner surface of the subspace with an essentially constant contact force. The mechanism thus generates a kind of counterforce to a contraction or relaxation force of the heart muscle forming the inner surface of the cavity. This counterforce can be used in particular to acquire, for example, the local force input transmitted from the heart muscle to the respective carrier Tₙ. For this purpose, the carriers Tₙ are advantageously equipped with a sensor system which makes it possible to acquire a local force input.

Furthermore particularly advantageously, the mechanism has a tube piece or hose piece which is arranged on the end part of the catheter so as to be axially longitudinally displaceable along the catheter and which is designed such that in the second state it is displaced axially distally, so that the carriers Tₙ and, if provided, existing devices for ablation and/or injection and/or for taking samples come to rest within the tube piece or hose piece, and is displaced axially proximally in the first state, so that the carriers Tₙ and possibly provided devices for ablation and/or injection and/or for sample taking come to rest outside the two-piece or hose piece and can freely unfold and can be used accordingly.

The mechanism is advantageously embodied such that the carriers Tₙ together with any possibly provided device(s) for ablation and/or injection and/or sample taking are retracted into the catheter in the second state. In this respect, the carrier Tₙ and the possibly provided device are embodied to be axially longitudinally displaceable in relation to a longitudinal axis of the catheter. In this example embodiment, a distal catheter lock of the cardiac catheter is designed to accommodate the carrier Tₙ and the device in the second state.

One particularly advantageous embodiment variant of the cardiac catheter is distinguished in that: N=3 and the orientations of the cross beams QT_{n,k} of the various longitudinal beams LTₙ differ from one another in the first state by 120 in each case. The alignments of the cross beams QT_{n,k} of the various longitudinal members LTₙ are particularly advantageously variably adjustable with respect to one another, so that various electrode configurations can be used for the measurement. For this purpose, the carriers Tₙ at the distal end of the cardiac catheter are particularly advantageously rotatable about their respective longitudinal axes LAₙ. Different relative configurations of the electrodes E_{n,m} arranged on various carriers Tₙ can thus be set in relation to one another.

One advantageous refinement of the cardiac catheter is distinguished in that the electrodes E_{n,m} have at least two different sizes of contact surfaces. The electrodes E_{n,m} are applied with their contact surfaces to the inner surface of the subspace of the heart in the first state.

The carriers Tₙ advantageously have a number of Mₙ first electrical lines for the individual contacting of the respective electrodes E_{n,m}, which are made of an electrically conductive, elastic polymer-based material.

The electrodes E_{n,m} advantageously have only two different sizes G1 and G2 of contact surfaces, wherein: G2≥2*G1.

One advantageous refinement of the cardiac catheter is distinguished in that the electrodes E_{n,m} have at least two different shapes of contact surfaces. The electrodes E_{n,m} advantageously have two different shapes F1 and F2 of contact surfaces, wherein the following is true: the shape F1 is circular and the shape F2 has a longitudinal extension which is greater than a perpendicular extension of the shape F2.

A first set of the electrodes E_{n,m} advantageously consists of a first material and a second set of the electrodes consists of a second material different therefrom. For example, the first set consists of gold electrodes and the second set consists of platinum electrodes.

One advantageous refinement of the proposed cardiac catheter is characterized in that the electrodes E_{n,m} of each cross beam QT_{n,k} have identical sizes of contact surfaces, wherein the sizes of contact surfaces of the electrodes E_{n,m} of successive cross beams QT_{n,k} on a longitudinal beam LTₙ alternate between two sizes of the contact surfaces, i.e., the sizes G1 and G2 of the electrodes of successive lines alternate as follows: QT_{n,k=1}=G1, QT_{n,k=2}=G2, QT_{n,k=3,m,n}=G1, . . . .

A distance between two adjacent electrodes E_{n,m} on a cross beam QT_{n,k} is advantageously constant for all electrodes E_{n,m} on this cross beam QT_{n,k}. The electrodes E_{n,m} advantageously consist of a metal or a metal alloy or of an electrically conductive polymer-based material or of carbon.

The electrodes E_{n,m} are used, on the one hand, to decrease electrical potentials, on the other hand, all or some selected electrodes E_{n,m} can also be used for the local application of current or voltage surges. The electrodes E_{n,m} are controlled accordingly for this purpose. In the region of its distal end, the catheter advantageously includes one or more electrodes, which are designed to deliver local electrical stimuli into the inner surface of the subspace.

Each carrier Tₙ advantageously has an electrical connecting element which is connected, on the one hand, via M electrical lines to the individual electrodes E_{n,m} of the carrier Tₙ, and on the other hand, is connectable to an evaluation unit via one or more electrical lines extending in the catheter. The connecting element advantageously includes an electrical amplifier and/or multiplexer and/or electrical filter.

At least one of the carriers Tₙ advantageously includes one or more additional sensors for acquiring one or more additional physical or chemical or biological parameters. The additional physical or chemical or biological parameter is advantageously a mechanical force and/or a mechanical torque acting externally on the carrier Tₙ, in particular from the inner surface of the subspace, a temperature, a concentration of a specified substance, a light signal, in particular a fluorescence signal, or a parameter dependent thereon.

Multiple additional sensors of this type are advantageously provided on at least one of the carriers T_{n,k} These sensors are advantageously arranged in such a way that a planar distribution of the respective parameter or parameters can be acquired. The measurement signals acquired by these sensors are advantageously conducted in the respective carrier Tₙ by elastic electrical lines to an interface which is advantageously arranged at the root of the respective carrier T_{n,k} From there, at least one electrical line (advantageously a metallic electrical line) is led to the proximal end of the catheter and provides sensor signals from the sensors for an evaluation unit there.

Further advantages, features, and details will be apparent from the following description, in which at least one example embodiment is described in detail, with reference to the drawings where appropriate. The same, similar, and/or functionally identical parts are provided with the same reference numerals.

### BRIEF DESCRIPTION OF THE DRAWINGS

In the drawings:

FIG. 1 shows a schematic illustration of a view of a distal end of a catheter according to the invention having a number N=3 carriers Tₙ, with n=1, 2, 3, from a position along the catheter towards the distal end of the catheter. In the present case, the catheter itself would extend perpendicularly from the plane of the sheet in the direction of its proximal end;

FIG. 2 shows a schematic side view of the distal end part of the catheter, having carriers that are in the second state;

FIG. 3 shows a schematic illustration of an arrangement according to the invention of electrodes E_{n,m} on a surface of a carrier Tₙ;

FIG. 4 shows a schematic illustration of a 3D view of a distal end of a catheter according to the invention in a second version, having a number N=3 of carriers Tₙ with n=1, 2, 3;

FIG. 5 shows a schematic illustration of a top view of the catheter shown in FIG. 4, wherein the longitudinal beams LTₙ each assume an angle of 120° in relation to one another;

FIG. 6 shows a schematic illustration of a top view of the catheter shown in FIG. 4, wherein the longitudinal beams LTₙ each assume an angle of 90° in relation to one another at their connection to the catheter;

FIG. 7 shows a schematic illustration of a top view of the catheter shown in FIG. 4, wherein the longitudinal beams LTₙ each assume an angle of 45° in relation to one another by rotation of the longitudinal beams at their connection to the catheter;

FIG. 8 shows a schematic illustration of a side view of the catheter shown in FIG. 4;

FIG. 9 shows a schematic illustration of a 3D view of the catheter shown in FIG. 4 having an inflatable body 108; and

FIG. 10 shows a schematic illustration of a top view of the catheter shown in FIG. 9.

### DETAILED DESCRIPTION

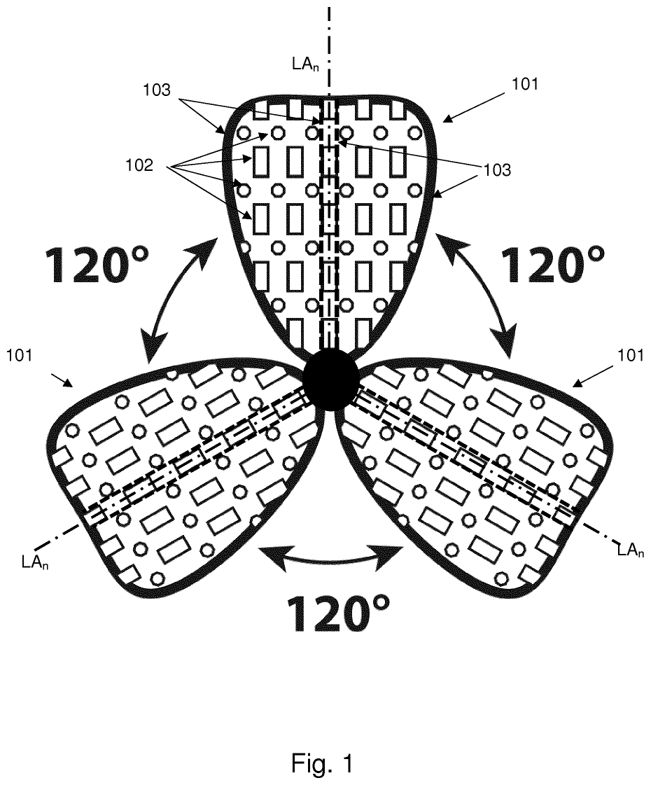

FIG. 1 shows a schematic illustration of a view of a distal end of a catheter according to the invention having a number N=3 of carriers Tₙ, with n=1, 2, 3, from a viewing position along the catheter towards the distal end of the catheter. In the present case, the catheter itself would extend perpendicularly from the plane of the sheet in the direction of its proximal end. In the present case, the catheter is shown in a first state, as would result if the carriers Tₙ were to unfold at the distal end of the catheter freely (elastic neutral state), i.e., undisturbed by external force actions, for example, due to contacts with the inner surface of the subspace of the heart.

The cardiac catheter shown has a number N=3 of elastic individual carriers Tₙ 101 with n=1, 2, 3, each of which is connected to a distal end region of the cardiac catheter. The catheter itself is represented by the black dot in the middle of the carrier Tₙ.

The longitudinal axes LAₙ of the carriers Tₙ 101 each enclose an angle of 120° in relation to one another. The three carriers Tₙ 101 each consist in the present case of an elastic membrane element MEM, wherein the membrane element MEM here has three layers: an upper layer on which the electrodes E_{n,m} 102 (shown in each case as cuboids and circles) are arranged, a lower layer, and a middle layer, in which electrical elastic first lines (not shown) for contacting the electrodes E_{n,m} 102 extend, wherein spacers are arranged in the middle layer to space apart upper layer and lower layer at a distance A, and the middle layer includes first electrical lines (not shown) made of an elastic, electrically conductive polymer for individually contacting the electrodes E_{n,m} 102.

The first lines connect the electrodes E_{n,m} 102 to an interface (not shown) on the catheter which connects the first electrical lines individually to second electrical lines, which are each insulated gold lines and which lead to the proximal end of the catheter. At the proximal end of the catheter, the second electrical lines are connectable to a corresponding evaluation device.

In the present case, the carriers Tₙ 101 each include a number Mₙ=45 of electrodes E_{n,m} 102, wherein the electrodes E_{n,m} 102 are arranged in K=9 lines R_{k,m,n} on the outside of the upper layer of the respective carrier Tₙ 101, with k=1, 2, 3. The lines R_{k,m,n} extend in the elastic neutral state shown (with a stretched-out support structure) perpendicular to the respective longitudinal axis LAₙ.

Furthermore, the catheter includes a mechanism configured so that the carriers Tₙ 101, after an insertion of the distal end region of the cardiac catheter into a subspace of a heart, can be reversibly brought from a first state into a second state, wherein the mechanism spreads out the carriers Tₙ 101 in the first state such that the electrodes E_{n,m} 102 are each applied to an inner surface of the subspace, and the mechanism closes the carriers Tₙ 101 in the second state such that the catheter having the carriers Tₙ 101 is removable from the subspace.

In the present case, the mechanism consists of a support structure, which is integrated into each of the individual carriers Tₙ 101, and a mechanical linkage (not shown) consisting of a mechanical connection of the three carriers, which enables the supports to “unfold” and using which “folding together” of the carriers is implementable. In this example embodiment, the support structure consists of wires 103 made of a shape memory material, in particular of nitinol. The wires 103 extend along the circumferential edge of the carriers Tₙ (thick black circumferential lines) and two wires 103 (thick dashed lines) along the longitudinal axis LAₙ of the respective carriers Tₙ. The supporting structure is preformed/conditioned in such a way that after a mechanical release it causes the carrier Tₙ to “unfold”. The mechanical release is advantageously carried out by an operator when the distal end of the catheter, i.e., specifically the carriers Tₙ, are introduced into the subspace of the heart to be examined.

The mechanical linkage (not shown) is designed here as a kind of “cable pull” in such a way that the spacing apart of the distal ends of the carriers Tₙ can be variably adjusted thereby. If the “cable pull” is “pulled”, the distal ends of the carriers Tₙ are pulled together, i.e., the carriers are therefore folded in and thus brought into the second state. If the “cable pull” is released, the carriers Tₙ unfold and assume a shape preformed by the support structure made of shape memory material (first state).

The conditioning of the support structure advantageously has the effect that the electrodes E_{n,m} 102 arranged on the upper layer are also applied to or are pressed against the inner surface of the subspace of the heart with a predetermined driving force even when the heart is beating.

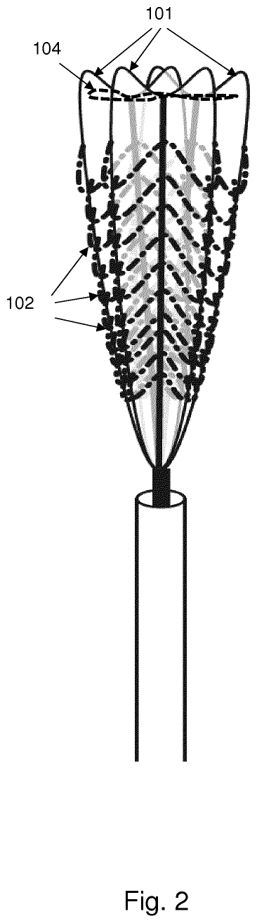

FIG. 2 shows a schematic side view of the distal end part of the catheter shown in FIG. 1, having carriers Tₙ which are in the second state. The cable pull 104 can be seen schematically, which causes the three carriers Tₙ to “fold up” when pulled together. If the cable pull 104 is “released”, the carriers Tₙ 101 thus unfold due to the support structures made of shape memory material.

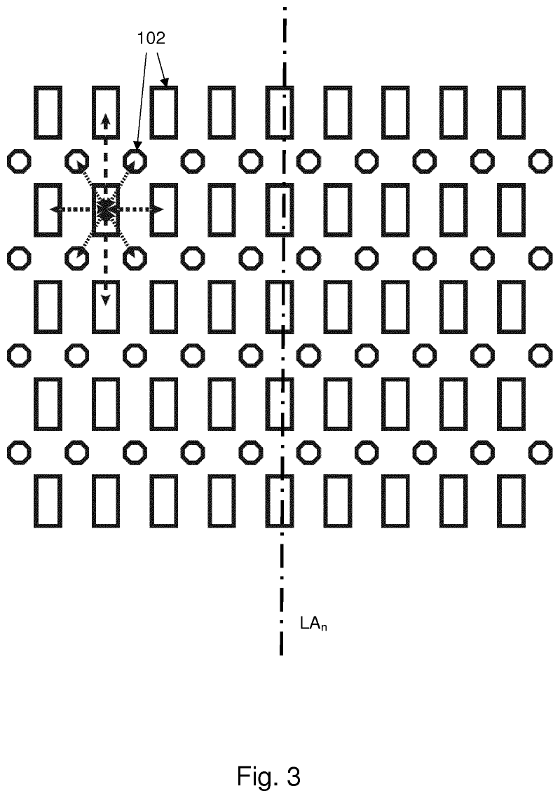

FIG. 3 shows a schematic illustration of an arrangement according to the invention of electrodes E_{n,m} on a surface of an elastic flat carrier Tₙ. It can be seen that the electrodes E_{n,m} are each aligned in rows perpendicular to a longitudinal axis LAₙ of the carrier Tₙ. Furthermore, it can be clearly seen that the electrodes E_{n,m} of a row each have the same shape of the contact surfaces and the same size of the contact surfaces. Furthermore, the electrodes E_{n,m} of a row are each equally spaced from one another. The electrodes E_{n,m} of successive rows alternately include electrodes E_{n,m} having round smaller contact surfaces and electrodes E_{n,m} having rectangular larger contact surfaces. Individual contacting of the electrodes E_{n,m} enables a large number of evaluations of differential potentials, as are indicated in FIG. 3 for an electrode on the basis of the arrows shown. The catheter preferably includes an electrode around which blood flows and which decreases a reference potential. The time-dependent potentials ascertained at the individual electrodes E_{n,m} can be determined against this reference potential.

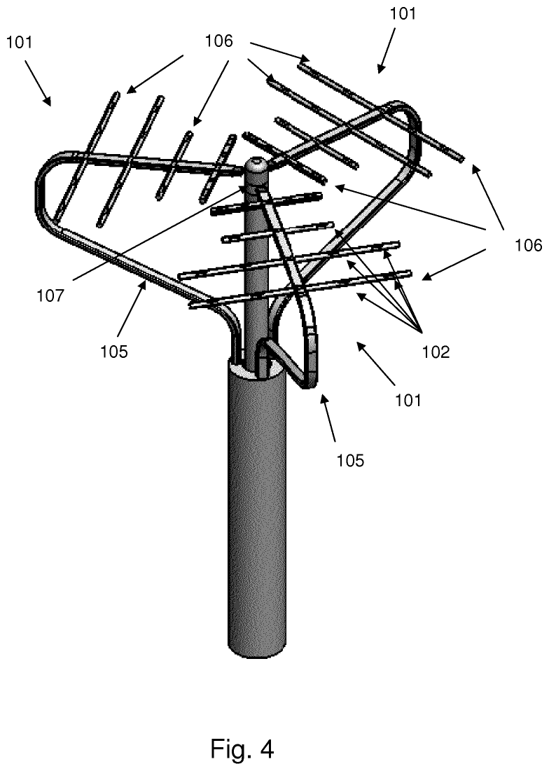

FIG. 4 shows a schematic illustration of a 3D view of a distal end of a cardiac catheter according to the invention in a second version, having a number N=3 of carriers Tₙ with n=1, 2, 3. The cardiac catheter includes:
a number N=3 of elastic individual carriers Tₙ 101 with n=1, 2, . . . , N=3, wherein the carriers Tₙ 101 each include an elastic longitudinal beam LTₙ 105 made of a shape memory alloy, the proximal end of which connects the respective carrier Tₙ 101 to a distal end region of the cardiac catheter and the distal end of which is a free end,
a longitudinal axis LAₙ extending along the respective longitudinal beam LTₙ 105,
a number Kₙ=4 of elastic cross beams QT_{n,k} 106, and
a number Mₙ=18 of electrodes E_{n,m} 102, with m=1, 2, . . . , Mₙ=18, wherein the electrodes E_{n,m} 102 are arranged on a respective outside of the respective cross beams QT_{n,k} 106, with k=1, 2, . . . Kₙ.

Furthermore, each carrier Tₙ include a mechanism configured so that the carriers Tₙ 101, after an insertion of the distal end region of the cardiac catheter into a subspace of a heart, can be reversibly brought from a first state into a second state, wherein the mechanism spreads out the carriers Tₙ 101 in the first state such that the electrodes E_{n,m} 102 are each applied to an inner surface of the subspace, the cross beams QT_{n,k}106 extend perpendicular to the respective longitudinal beam LTₙ 105 and/or perpendicular to the longitudinal axis LAₙ and the mechanism closes the carriers Tₙ 101 in the second state such that the catheter having the carriers Tₙ 101 is removable from the subspace.

Finally, the catheter includes a central operable device 107 for ablation and/or for injection and/or for taking samples.

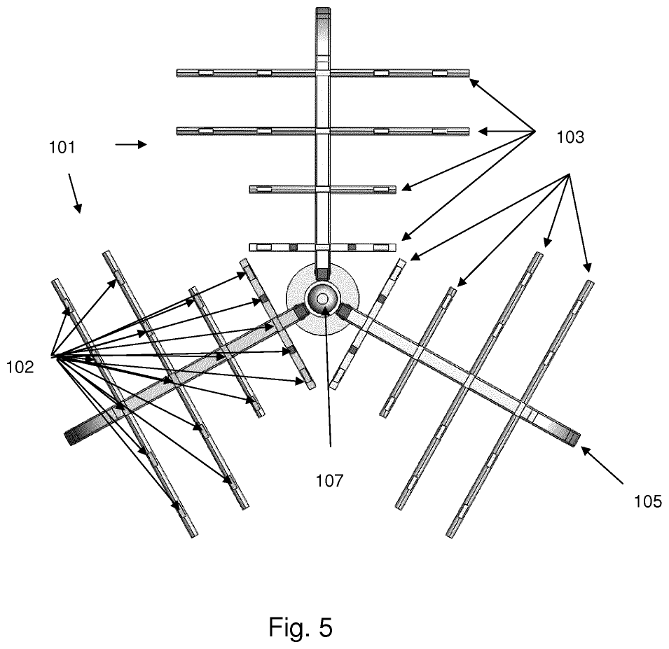

Because longitudinal beams LTₙ can be rotated about their longitudinal axis at the connection point, the carriers Tₙ can be arranged differently in relation to one another. FIGS. 5 to 7 show different configurations corresponding thereto. The distal ends of the longitudinal members LTₙ are free ends here, and in particular are not connected to one another or to the central device 107.

FIG. 5 shows a schematic illustration of a top view of the catheter shown in FIG. 4, wherein the longitudinal beams LTₙ each assume an angle of 1200 in relation to one another. The device 107 for ablation and/or for injection and/or for taking samples can be seen centrally. Furthermore, the 3×18 electrodes E_{n,m} 102 arranged on the outside of the cross beams QT_{n,k} 106 can be seen, which essentially have the same rectangular shape, with the exception of six electrodes E_{n,m} on the respective distal cross beams QT_{n,k} 106, which have a different square shape.

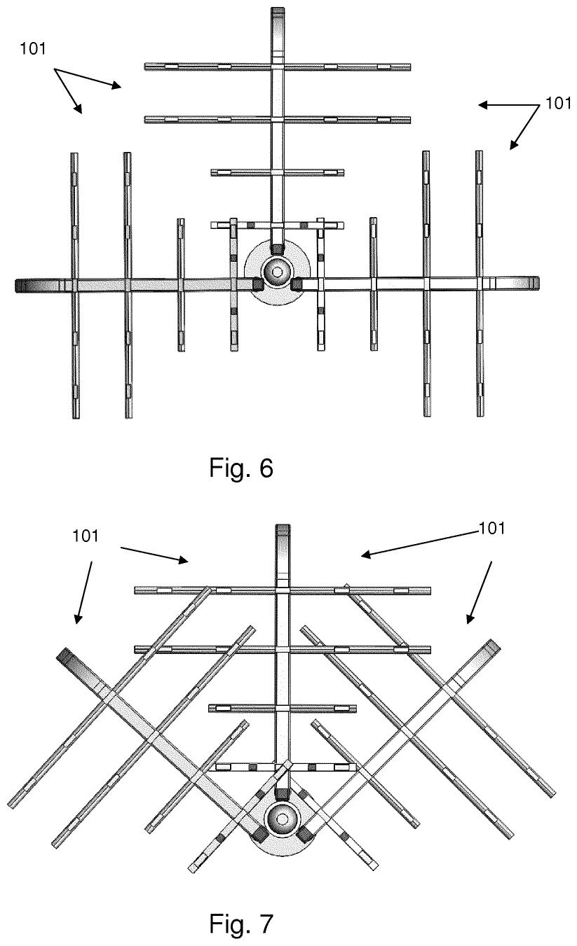

FIG. 6 shows a schematic illustration of a top view of the catheter shown in FIG. 4, wherein the longitudinal beams LTₙ 105 each assume an angle of 900 in relation to one another by rotation of the longitudinal beams at their connection to the catheter.

FIG. 7 shows a schematic illustration of a top view of the catheter shown in FIG. 4, wherein the longitudinal beams LTₙ 105 each assume an angle of 450 in relation to one another by rotation of the longitudinal beams at their connection to the catheter.

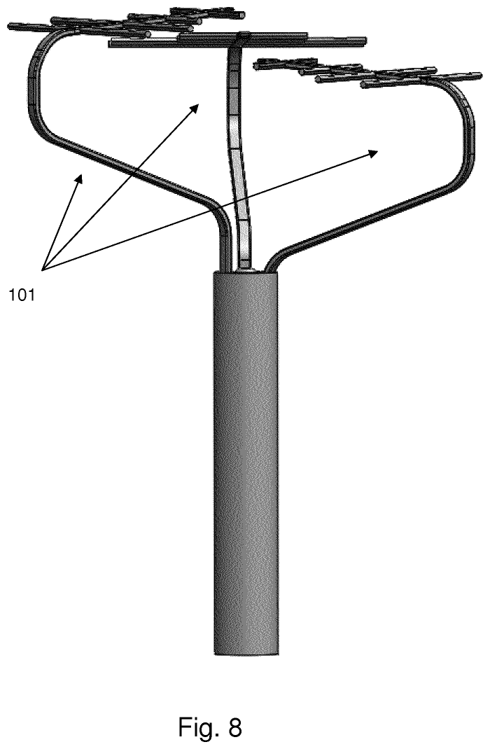

FIG. 8 shows a schematic illustration of a side view of the catheter shown in FIG. 4.

It can be clearly seen that the longitudinal beams LTₙ 105 in the first state are advantageously conditioned in such a way that configurations are settable in which the cross beams QT_{n,k} 106 can overlap. In the example shown, the longitudinal beams LTₙ 105 are vertically spaced apart from the respective cross beams QT_{n,k} 106, so that overlapping configurations are settable, as shown, for example, in FIG. 7.

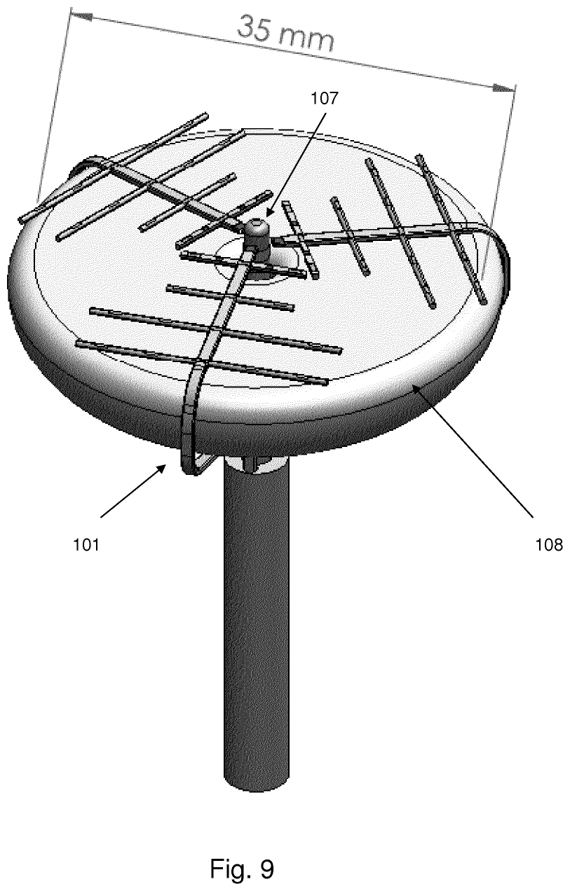

FIG. 9 shows a schematic illustration of a 3D view of the catheter shown in FIG. 4 having an inflatable body 108.

After the distal end part of the catheter has been inserted into a cavity in the heart, the carriers Tₙ are unfolded and, by corresponding rotation of the carriers Tₙ around the local longitudinal axis of the longitudinal beams LTₙ at the linkage point, the desired relative configuration of the carriers Tₙ is set. After the desired relative configuration of the carriers has been set, the inflatable body 108 is filled with a fluid so that it expands and presses the outside of the cross beams QT_{n,k} 106 with the electrodes E_{n,m} 102 arranged thereon against the inside of the cavity in the heart.

After completion of the measurements or after completion of measures using the device 107, the inflatable body 108 is first emptied of fluid, so that the carriers Tₙ are freely movable again.

The mechanism advantageously includes a tube piece or hose piece (not shown) which is arranged on the end part of the catheter so as to be axially longitudinally displaceable along the catheter and which is designed such that in the second state it is displaced axially distally, so that the carriers Tₙ and possibly provided devices for ablation and/or injection and/or for taking samples come to rest within the tube piece or hose piece, and is displaced axially proximally in the first state, so that the carriers Tₙ and possibly provided devices for ablation and/or injection and/or for sample taking come to rest outside the two-piece or hose piece and can freely unfold and can be used accordingly.

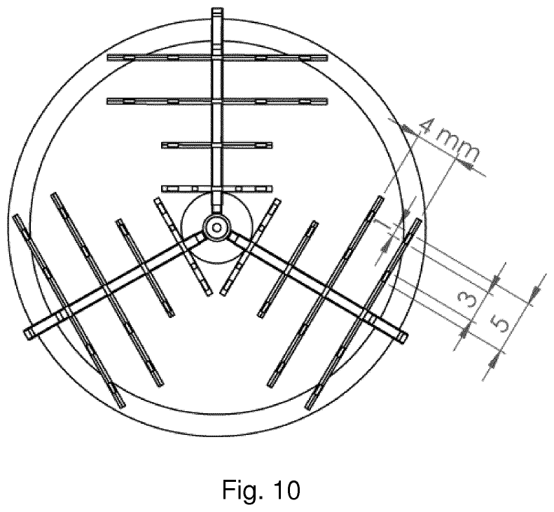

FIG. 10 shows a schematic illustration of a top view of the catheter shown in FIG. 9 having dimensions in [mm].

Although the invention has been further illustrated and described in detail by way of preferred embodiments, the invention is not limited by the disclosed examples, and other variations can be derived therefrom by a person skilled in the art without departing from the scope of protection of the invention. It is therefore clear that a multitude of possible variations exists. It is also clear that embodiments mentioned by way of examples are really only examples, which are not to be construed in any way as limiting the scope of protection, the possible applications, or the configuration of the invention. Rather, the foregoing description and the description of the figures enable a person skilled in the art to implement the example embodiments, wherein a person skilled in the art in the knowledge of the disclosed inventive concept may make various changes, for example as to the function or arrangement of individual elements cited in an example embodiment, without departing from the scope of protection as defined by the claims and their legal equivalents, such as more extensive explanations in the description.

### LIST OF REFERENCE SIGNS

101 carrier Tₙ
102 electrodes E_{n,m}
103 wires made from a shape memory material
104 cable pull
105 longitudinal beam LTₙ
106 cross beam QT_{n,k}
107 ablation tool
108 inflatable body

1. A cardiac catheter having electrodes, said catheter comprising a number N of elastic individual carriers Tₙ with n=1, 2, . . . , N and Nϵ{2, 3, . . . , 10}, which are each connected to a distal end region of the cardiac catheter, wherein the carriers Tₙ each have a longitudinal axis LAₙ, a number Mₙ of electrodes E_{n,m} per carrier Tₙ, with m=1, 2, . . . , Mₙ and Mₙ≥5, wherein the electrodes E_{n,m} are arranged in lines R_{k,m,n} on an outside of the respective carrier Tₙ, with k=1, 2, . . . K and K≥3, the lines R_{k,m,n} extend perpendicular to the longitudinal axis LAₙ, and furthermore the carriers comprise a mechanism by means of which said carriers Tₙ, after an insertion of the distal end region of the cardiac catheter into a subspace of a heart, are capable of being reversibly brought from a first state into a second state, wherein the mechanism spreads out the carriers Tₙ in the first state such that the electrodes E_{n,m} are each applied to an inner surface of the subspace, and the mechanism closes the carriers Tₙ in the second state such that the catheter having the carriers Tₙ is removable from the subspace.

2. The cardiac catheter as claimed in claim 1, wherein the carriers Tₙ each comprise:
an elastic longitudinal beam LTₙ, the proximal end of which connects the carrier Tₙ to a distal end region of the cardiac catheter and the distal end of which is a free end;
a longitudinal axis LAₙ extending along the respective longitudinal beam LTₙ;
a number Kₙ of elastic cross beams QT_{n,k}; and
a number Mₙ of electrodes E_{n,m}, with m=1, 2, . . . , Mₙ and Mₙ≥5, wherein the electrodes E_{n,m} are arranged on a respective outside of the respective cross beams QT_{n,k}, with k=1, 2, . . . Kₙ and Kₙ≥3,
wherein the mechanism spreads out the carriers Tₙ in the first state such that the cross beams QT_{n,k} extend perpendicular to the respective longitudinal beam LTₙ and/or perpendicular to the longitudinal axis LAₙ.

3. The cardiac catheter as claimed in claim 1, wherein the carriers Tₙ each comprise at least three markers of a position acquisition system.

4. The cardiac catheter as claimed in claim 2, wherein the carriers Tₙ each comprise a flat elastic membrane MEM, which connects the cross beams QT_{n,k} and the longitudinal beams LTₙ of the respective carrier Tₙ.

5. The cardiac catheter as claimed in claim 2, wherein the cross beams QT_{n,k} and/or the longitudinal beam LTₙ consist of a shape memory alloy.

6. The cardiac catheter as claimed in claim 2, wherein the cross beams QT_{n,k} and/or the longitudinal beam LTₙ comprise fluid chambers, which are capable of being individually filled with fluid or emptied of fluid.

7. The cardiac catheter as claimed in claim 2, wherein the longitudinal beams LTₙ are connected to the distal end region of the cardiac catheter in such a way that they are rotatable.

8. The cardiac catheter as claimed in claim 1, wherein the carriers Tₙ have a number Mₙ of first electrical lines, made of an electrically conductive, elastic polymer-based material, for the individual contacting of the respective electrodes E_{n,m}.

9. The cardiac catheter as claimed in claim 4, wherein the flat elastic membrane MEM comprises the following three elastic layers:
an upper layer on which the electrodes E_{n,m} are arranged;
a lower layer; and
a middle layer in which electrical elastic first lines for contacting the electrodes E_{n,m} extend,
wherein spacers are arranged in the middle layer in order to space apart the upper layer and lower layer at a distance A, and the middle layer comprises a volume through which a fluid can flow.

10. The cardiac catheter as claimed in claim 10, wherein the mechanism is embodied and configured such that the carriers Tₙ and the electrodes E_{n,m} are pressed against the inner surface of the subspace in the first state with a predetermined contact force.

11. The cardiac catheter as claimed in claim 1, wherein the mechanism has an inflatable body, which is capable of being filled and emptied, and which is capable of being filled with a fluid in the first state and thereby presses the carriers Tₙ outward from an inside of the respective carrier Tₙ.

12. The cardiac catheter as claimed in claim 1, wherein the mechanism has a tube piece or hose piece which is arranged on the end part of the catheter so as to be axially longitudinally displaceable along the catheter and which is designed such that in the second state it is displaced axially distally, so that the carriers Tₙ come to rest within the tube piece or hose piece, and is displaced axially proximally in the first state, so that the carriers Tₙ can freely unfold.

13. The cardiac catheter as claimed in claim 1, wherein the electrodes E_{n,m} only have two different sizes G1 and G2 of contact surfaces, wherein: G2≥2*G.

14. The cardiac catheter as claimed in claim 1, wherein at least one of the carriers Tₙ comprises one or more additional sensors for acquiring an additional physical or chemical or biological parameter.
